(19) United States
(12) Patent Application Publication
(10) Pub. No.: US 2004/0014060 A1
(43) Pub. Date: Jan. 22, 2004

(54) Doped nanoparticles as biolabels

(76) Inventors: Werner Hoheisel, Koln (DE); Christoph Petry, Krefeld (DE); Markus Haase, Hamburg (DE); Karsten Riwotzki, Heidelberg (DE); Kerstin Bohmann, Koln (DE)

Correspondence Address: KURT BRISCOE, NORRIS, MCLAUGHLIN & MARCUS, P.A., 220 EAST 42ND STREET, 30TH FLOOR, NEW YORK, NY, 10017, US

(21) Appl. No.: 10/275,355

(22) PCT Filed: Apr. 23, 2001
(86) PCT No.: PCT/EP01/04545

(30) Foreign Application Priority Data
May 5, 2000 (DE) 100 21 674.9
Feb. 12, 2001 (DE) 101 06 643.0

(51) Int. Cl. C12Q 1/68; G01N 33/53; C07H 21/04; C07K 14/47; C07K 16/46

(52) U.S. Cl. 435/6; 435/7.1; 530/400; 536/24.3

(57) ABSTRACT

The invention relates to a simple detection probe containing luminescent inorganic doped nanoparticles (l.i.d nanoparticles) which can be detected after excitement by a source of radiation by their absorption and/or scattering and/or diffraction of the excitement radiation or by emission of fluorescent light, and whose surface is prepared in such a way that affinity molecules for detecting a biological or other organic substance can couple with this prepared surface.

The present invention relates to a detection probe for biological applications, which comprises luminescent inorganic doped nanoparticles (lid nanoparticles).

The use of markers in biological systems for marking or monitoring specific substances has been an established tool in medical diagnostics and biotechnological research for decades. Such markers are applied in particular in flow cytometry, histology, in immunoassays or in fluorescence microscopy, in the latter for studying biological and nonbiological materials.

The marker systems most common in biology and biochemistry are radioactive isotopes of iodine, phosphorus and of other elements and also enzymes such as horseradish peroxidase or alkaline phosphatase, the detection of which requires specific substrates. Moreover, markers which are increasingly being used are fluorescent organic dye molecules such as fluorescein, Texas Red or Cy5, which are attached selectively to a particular biological or other organic substance. Depending on the system used, usually a further linker molecule or a combination of further linker molecules or affinity molecules between the substance to be detected and the marker, which has the specific affinity required in order to unambiguously recognize the substance to be detected, is required. The technique required for this is known and is described, for example, in “Bioconjugate Techniques”, G. T. Hermanson, Academic Press, 1996 or in “Fluorescent and Luminescent Probes for Biological Activity. A Practical Guide to Technology for Quantitative Real-Time Analysis”, Second Edition, W. T. Mason, ed., Academic Press, 1999. After external, usually electromagnetic, excitation of the marker, said marker will then indicate via the emission of fluorescent light the presence of the biological or other organic substances bound to said marker.

The fluorescent organic dye molecules which represent the current state of the art have the disadvantage of being irreversibly damaged or destroyed, in particular in the presence of oxygen or free radicals and sometimes after just a few million light absorption/light emission cycles. Thus, their stability to incident light is frequently insufficient for many applications. Furthermore, the fluorescent organic dye molecules may also have a phototoxic effect on the biological environment.

Another disadvantage of the fluorescent organic dyes are their broad emission bands which frequently have an additional extension at the long-wave end of the fluorescence spectrum. This impairs a “multiplexing”, i.e. the simultaneous identification of a plurality of substances labeled with in each case different fluorescent dyes, due to the, in this case, partially overlapping emission bands, and severely limits the number of different substances detectable in parallel. Another disadvantage of using a plurality of organic fluorescent dyes simultaneously are the relatively narrow spectral excitation bands within which the dye can be excited. In order to be able to excite all dyes efficiently, a plurality of light sources, generally lasers, or a complicated optical design using a source of white light and a suitable arrangement of color filters must therefore be used.

Fluorescent inorganic semiconductor nanocrystals have been proposed as alternative markers to the fluorescent organic dyes. U.S. Pat. No. 5,990,470 and the PCT applications WO 00/17642 and WO 00/29617 disclose that fluorescent inorganic semiconductor nanocrystals which are members of the class of II-VI or III-V semiconductor compounds and which may, subject to certain conditions, also comprise elements of the fourth main group of the Periodic Table can be used as fluorescent markers in biological systems. The emission wavelength of the fluorescent light of the semiconductor nanocrystals can be set in the visible and near infrared spectral range by varying the size of said semiconductor nanocrystals, utilizing the “quantum size effect”. The exact position of the emission wavelength depends on the solid-state band gap between conduction band and valence band of the semiconductor material chosen and is determined by the particle size and/or by the distribution thereof. Semiconductor nanocrystals and their use as biological markers is furthermore disclosed in Warren C. W. Chan and Shuming Nie, Science, Vol. 281, 1998, pages 2016-2018 and Marcel Bruchez Jr., Mario Moronne, Peter Gin, Shimon Weiss, A. Paul Alivisatos, Science, Vol. 281, 1998, pages 2013-2016.

Disadvantageously, the semiconductor nanocrystals must be prepared with the highest precision and thus cannot be produced easily. Since the emission wavelength of the fluorescent light depends on the size of the semiconductor nanocrystals, a narrow bandwidth of the fluorescent light which is composed of fluorescent light emission from a multiplicity of individual semiconductor nanocrystals requires a very narrow size distribution of said semiconductor nanocrystals. In order to ensure the narrow fluorescent light bandwidth required for multiplexing, the individual semiconductor nanocrystals may differ in size by only a few Angstrom, i.e. by only a few monolayers. This makes great demands on the synthesis of semiconductor nanocrystals. In addition, the semiconductor nanocrystals were observed as having relatively weak quantum yields, due to radiationless electron-hole pair recombinations on the surface of the semiconductor nanocrystals. For this reason, a complicated core-shell structure was proposed, the core comprising the actual semiconductor material and the shell comprising a further semiconductor material with a larger band gap (e.g. CdS or ZnS) which is epitaxially grown over the core, if possible. In order for these core-shell particles to be able to attach to the biological material to be detected, another, thin shell which preferentially comprises silica glass (SiOₓ, x=1-2) was additionally applied (U.S. Pat. No. 5,990,479, WO 99/121934, EP 1034234, Peng et al., Journal of the American Chemical Society, Vol. 119, 1997, pages 7019-7029). A multiple core-shell structure of this kind includes further relatively complicated synthesis steps. Another disadvantage is the fact that the majority of the semiconductor nanocrystals known from the literature and nearly all of those used in practice up until now contain elements which must be classified as toxic, such as, for example, cadmium, selenium, tellurium, indium, arsenic, gallium or mercury.

Furthermore, it is possible to use colloids of noble metals such as gold or silver as probes for detecting specific biological substances. The surfaces of said colloids have been modified such that conjugation with biomolecules is possible. The colloids are detected via measurement of light absorption or of the elastically scattered light after irradiation of white light. Thus, by exciting the surface plasma resonance of the metal particles whose wavelength is specific for the material and for the particle size, it is possible to identify specifically a particular class of particles and thus also the corresponding conjugates (S. Schultz, D. R. Smith, J. J. Mock, D. A. Schultz; Proceedings of the National Academy of Science, Vol. 97, Issue 3, Feb. 1, 2000, pages 996-1001). The detection is very sensitive in the large absorption cross section and scattering cross section. However, the disadvantage of this solution is the relatively small selection of available working wavelengths so that true multiplexing is possible only with limitations. Moreover, the light-scattering efficiency depends very strongly on the material and on the particle size so that the detection sensitivity for a biomolecule to be detected depends on the material, but to a great extent on the size and thus on the scattering color of the metal particle acting as reporter.

The patents U.S. Pat. No. 4,637,988 and U.S. Pat. No. 5,891,656 disclose the possibility of using metal chelates having a metal ion of the lanthanide series as fluorescent markers. This system is advantageous in that the states excited by the absorption of light have long lifetimes which extend up to the millisecond range. This enables the reporter fluorescence to be detected in a time-resolved manner so that autofluorescent light can be virtually completely suppressed. However, these chelate systems often have the disadvantage of their luminescence being drenched in aqueous media which are required for most biological applications. Therefore, it is often necessary to separate chelates in an additional step from the substance actually to be detected and to transfer them to an anhydrous environment (I. Hemmilä, Scand. J. Clin. Lab. Invest. 48, 1988, pages 389-400). As a result, however, immunohistochemical studies are not possible, since the spatial information of the label is lost in the separation step.

The patents U.S. Pat. No. 4,283,382 and U.S. Pat. No. 4,259,313 disclose the possibility of using polymer (latex) particles in which metal chelates having a metal ion of the lanthanide series are embedded likewise as fluorescent markers.

Luminescent phosphors which have been used as coating material in fluorescent lamps or in cathode ray tubes for a long time were likewise used as reporter particles in biological systems. U.S. Pat. No. 5,043,265 discloses the possibility of detecting biological macromolecules coupled to luminescent phosphor particles by fluorescence measurement. It is stated that the phosphor particles should be smaller than 5 μm, preferably smaller than 1 μm. However, it is also stated that the fluorescence intensity of the particles rapidly decreases with decreasing diameter and the particles should therefore be larger than 20 nm and, preferably, even larger than 100 nm. The reason for this is apparently, inter alia, the method of preparing said particles. Starting from commercially available luminescent phosphors of around 5 μm in size, these are reduced to a size of less than 1 μm by ball-milling. Disadvantageously, this procedure leads to a broad particle size distribution and to a generally relatively high degree of agglomeration. Moreover, a large number of defects which may considerably reduce the quantum efficiency of the fluorescence radiation are probably introduced into the crystal structure of the particles. Another disadvantage is the fact that the particles disclosed in said invention, due to their size of usually several 100 nanometers and a broad size distribution, are excluded from many applications which involve marker mass and marker size, as is the case, for example, when staining cell components or monitoring substances.

U.S. Pat. No. 5,893,999 claims specific preparation methods for particular luminescent phosphors of between 1 nm and 100 nm in size, which are reportedly also useful for biological applications. In this application it is stated that the particles can be prepared by gas-phase syntheses (vaporization and condensation, RF thermal plasma process, plasma spraying, sputtering) and by hydrothermal syntheses. The disadvantages of these particles, in particular for applications in the fields of biology and biochemistry, are the high degree of agglomeration of the primary particles and thus to the large overall size of the agglomerates usable in practice and also the very broad size distribution of the particles used, all of which is inherently due to the preparation processes described. Moreover, both the degree of agglomeration and the broad size distribution are clearly visible in the electron micrographs included in the patent publication.

U.S. Pat. No. 5,674,698 discloses specific types of luminescent phosphors for use as biological labels. These are “upconverting phosphors” which have the property of emitting, via a two-photon process, light which has a shorter wavelength than the absorbed light. Using these particles makes it possible to work basically background-free, since this autofluorescence is very substantially suppressed. The particles are prepared by milling and subsequent heat treatment. The particle size is between 10 nm and 3 μm, preferably between 300 nm and 1 μm. Disadvantages here are again the large particle size and the broad size distribution due to the preparation process.

U.S. Pat. No. 5,891,361 and U.S. Pat. No. 6,039,894 disclose a preparation method for these “upconverting” luminescent phosphors, which does not involve milling. These are precipitation products which are converted to fluorescent phosphors of between 100 nm and 1 μm in size by partially reactive high-temperature aftertreatments in the gas phase. Here too, the disadvantages are again the large particle sizes and the broad size distribution, caused by the high temperatures during synthesis.

Scientific publications deal with the preparation of selected luminescent inorganic doped nanoparticles and with studies on the luminescence properties thereof. The published luminescent inorganic doped nanoparticles consist of oxides, sulfides, phosphates or vanadates, which are doped with lanthanides or else with Mn, Al, Ag or Cu. These luminescent inorganic doped nanoparticles fluoresce in a narrow spectral range due to their doping. A potential application is seen in their use as phosphors in cathode ray tubes or as luminescent substances in lamps. Inter alia, the preparation of the following luminescent inorganic doped nanoparticles has been published: YVO₄:Eu, YVO₄:Sm, YVO₄:Dy (K. Riwotzki, M. Haase; Journal of Physical Chemistry B; Vol. 102, 1998, pages 10129 to 10135); LaPO₄:Eu, LaPO₄:Ce, LaPO₄:Ce,Tb; (H. Meyssamy, K. Riwotzki, A. Komowski, S. Naused, M. Haase; Advanced Materials, Vol. 11, Issue 10, 1999, pages 840 to 844); (K. Riwotzki, H. Meyssamy, A. Kornowski, M. Haase; Journal of Physical Chemistry B Vol. 104, 2000, pages 2824 to 2828); ZnS:Tb, ZnS:TbF₃, ZnS:Eu, ZnS:EuF₃, (M. Ihara, T. Igarashi, T. Kusunoki, K. Ohno; Society for Information Display, Proceedings 1999, Session 49.3); Y₂O₃:Eu (Q. Li, L. Gao, D. S. Yan; Nanostructured Materials Vol. 8, 1999, pages 825 ff); Y₂SiO₅:Eu (M. Yin, W. Zhang, S. Xia, J. C. Krupa; Journal of Luminescence, Vol. 68, 1996, pages 335 ff.); SiO₂:Dy, SiO₂:Al, (Y. H. Li, C. M. Mo, L. D. Zhang, R. C. Liu, Y. S. Liu; Nanostructured Materials Vol. 11, Issue 3, 1999, pages 307 to 310); Y₂O₃:Tb (Y. L. Soo, S. W. Huang, Z. H. Ming, Y. H. Kao, G. C. Smith, E. Goldburt, R. Hodel, B. Kulkami, J. V. D. Veliadis, R. N. Bhargava; Journal of Applied Physics Vol. 83, Issue 10, 1998, pages 5404 to 5409); CdS:Mn (R. N. Bhargava, D. Gallagher, X. Hong, A. Nurrnikko; Physical Review Letters Vol. 72, 1994, pages 416 to 419); ZnS:Tb (R. N. Bhargava, D. Gallagher, T. Welker; Journal of Luminescence, Vol. 60, 1994, pages 275 ff.).

An overview of the known luminescent inorganic doped materials and their use as technical phosphors which are a few micrometers in size can be found in Ullmann's Encyclopedia of Industrial Chemistry, WILEY-VCH, 6^{th} edition, 1999, Electronic Release, Chapter “Luminescent Materials: 1. Inorganic Phosphors”. The review found there refers exclusively to the material classes which can be used for the applications described there and not to particular properties of these materials in the form of nanoparticles.

It is an object of the present invention to provide a detection probe for biological applications which comprises inorganic luminescent particles of a few nanometers in size and which does not have the above-described disadvantages of the markers known in the prior art.

The object of the invention is achieved by a detection probe for biological applications, comprising luminescent inorganic doped nanoparticles (lid nanoparticles).

Lid nanoparticles are doped with foreign ions in such a way that, after excitation using a radiation source, they can be detected material-specifically via absorption and/or scattering and/or diffraction of said radiation or via emission of fluorescent light. The lid nanoparticles can be excited by narrow-band or broadband electromagnetic radiation or by a particle beam. The particles are qualitatively and/or quantitatively detected by measuring a change in the absorption and/or scattering and/or diffraction of said radiation or by measuring material-specific fluorescent light or the change therein.

The lid nanoparticles have a virtually spherical morphology with expansions in the range from 1 nm to 1 μm, preferably in the range from 2 nm to 100 nm, particularly preferably in the range from 2 nm to below 20 nm and very particularly preferably between 2 nm and 10 nm. Expansions mean the maximum distance between two points located on the surface of an lid particle. The lid nanoparticles may also have an ellipsoid-like morphology or may be faceted, with expansions being within the abovementioned limits. In addition, the lid nanoparticles may also have a distinctive needle-like morphology with a width of from 3 nm to 50 nm, preferably from 3 nm to below 20 nm and with a length of from 20 nm to 5 μm, preferably from 20 nm to 500 nm. The particle size can be determined using the ultracentrifugation method or gel permeation chromatography method or by means of electron microscopy.

Materials suitable according to the invention for lid nanoparticles are inorganic nanocrystals whose crystal lattice (host material) is doped with foreign ions. Included herein are in particular all materials and material classes which are used as “phosphors”, for example, in phosphor screens (e.g. for electron ray tubes) or as coating material in fluorescent lamps (for gas discharge lamps), which phosphors are mentioned, for example, in Ullmann's Encyclopedia of Industrial Chemistry, WILEY-VCH, 6^{th} edition, 1999 Electronic Release, Chapter “Luminescent Materials: 1. Inorganic Phosphors”, and the luminescent inorganic doped nanoparticles known in the prior art cited above. In these materials, the foreign ions serve as activators of fluorescent light emission after excitation by UV light, visible light or IR light, X-rays or gamma rays or electron rays. In addition, a plurality of foreign ion types are incorporated into the host lattice of some materials in order to, on the one hand, generate activators for emission and, on the other hand, make excitation of the particle system more efficient, or in order to adjust the absorption wavelength by a shift to the wavelength of a given excitation light source (“sensitizers”). The incorporation of a plurality of types of foreign ions may also serve to specifically set up a particular combination of fluorescent bands which a particle is intended to emit.

The host material of the lid nanoparticles preferably comprises compounds of the XY type. In this connection, X is a cation of elements of the main groups 1a, 2a, 3a, 4a, of the transition groups 2b, 3b, 4b, 5b, 6b, 7b or of the lanthanides of the Periodic Table. In some cases, X may also be a combination or a mixture of said elements. Y may be a polyatomic anion comprising one or more element(s) of the main groups 3a, 4a, 5a, of the transition groups 3b, 4b, 5b, 6b, 7b and/or 8b and also elements of the main groups 6a and/or 7a. However, Y may also be a monoatomic anion of the main group 5a, 6a or 7a of the Periodic Table. The host material of the lid nanoparticles may also comprise an element of main group 4a of the Periodic Table. Elements of main groups 1a, 2a or of the group comprising Al, Cr, TI, Mn, Ag, Cu, As, Nb, Nd, Ni, Ti, In, Sb, Ga, Si, Pb, Bi, Zn, Co and/or elements of the lanthanides may serve as doping agent. Combinations of two or more of these elements at different relative concentrations to one another may also serve as doping material. The doping material concentration in the host lattice is between 10⁻⁵ mol % and 50 mol %, preferably between 0.01 mol % and 30 mol %, particularly preferably between 0.1 mol % and 20 mol %.

Preference is given to using sulfides, selenides, sulfoselenides, oxysulfides, borates, aluminates, gallates, silicates, germanates, phosphates, halophosphates, oxides, arsenates, vanadates, niobates, tantalates, sulfates, tungstates, molybdates, alkali halides and other halides or nitrides as host materials for the lid nanoparticles. Examples of these material classes together with the corresponding dopings are given in the following list (type B materials: A+B=host material and A=doping material):

LiI:Eu; NaI:TI; CsI:Tl; CsI:Na; LiF:Mg; LiF:Mg,Ti; LiF:Mg,Na; KMgF₃:Mn; Al₂O₃:Eu; BaFCl:Eu; BaFCl:Sm; BaFBr:Eu; BaFCl_{0.5}Br_{0.5}:Sm; BaY₂F₈:A (A=Pr, Tm, Er, Ce); BaSi₂O₅:Pb; BaMg₂Al₆O₂₇:Eu; BaMgAl₁₄O₂₃:Eu; BaMgA₁₀O₁₇:Eu; BaMgAl₂O₃:Eu; Ba₂P₂O₇:Ti; (Ba,Zn,Mg)₃Si₂O₇:Pb; Ce(Mg,Ba)Al₁₁O₁₉; Ce_{0.65}Tb_{0.35}MgAl₁₁O₁₉:Ce,Tb; MgAl₁₁O₁₉:Ce,Tb; MgF₂:Mn; MgS:Eu; MgS:Ce; MgS:Sm; MgS:(Sm,Ce); (Mg,Ca)S:Eu; MgSiO₃:Mn; 3.5MgO.0.5MgF₂.GeO₂:Mn; MgWO₄:Sm; MgWO₄:Pb; 6MgO.As₂O₅:Mn; (Zn,Mg)F₂:Mn; (Zn₄Be)SO₄:Mn; Zn₂SiO₄:Mn; Zn₂SiO₄:Mn,As; ZnO:Zn; ZnO:Zn,Si,Ga; Zn₃(PO₄)₂:Mn; ZnS:A (A=Ag, Al, Cu); (Zn,Cd)S:A (A=Cu, Al, Ag, Ni); CdBO₄:Mn; CaF₂:Mn; CaF₂:Dy; CaS:A (A=lanthanides, Bi); (Ca,Sr)S:Bi; CaWO₄:Pb; CaWO₄:Sm; CaSO₄:A (A=Mn, lanthanides); 3Ca₃(PO₄)₂.Ca(F,Cl)₂:Sb,Mₙ; CaSiO₃:Mn,Pb; Ca₂Al₂Si₂O₇:Ce; (Ca,Mg)SiO₃:Ce; (Ca,Mg)SiO₃:Ti; 2SrO.6(B₂O₃).SrF₂:Eu; 3Sr₃(PO₄)₂.CaCl₂:Eu; A₃(PO₄)₂Acl₂:Eu (A=Sr, Ca, Ba); (Sr,Mg)₂P₂O₇:Eu; (Sr,Mg)₃(PO₄)₂:Sn; SrS:Ce; SrS:Sm,Ce; SrS:Sm; SrS:Eu; SrS:Eu,Sm; SrS:Cu,Ag; Sr₂P₂O₇:Sn; Sr₂P₂O₇:Eu; Sr₄Al₁₄O₂₅:Eu; SrGa₂S₄:A (A=lanthanides, Pb); SrGa₂S₄:Pb; Sr₃Gd₂Si₆O₁₈:Pb,Mn; YF₃:Yb,Er; YF₃:Ln (Ln=lanthanides); YLiF₄:Ln (Ln=lanthanides); Y₃Al₅O₁₂:Ln (Ln=lanthanides); YAl₃(BO₄)₃:Nd,Yb; (Y,Ga)BO₃:Eu; (Y,Gd)BO₃:Eu; Y₂Al₃Ga₂O₁₂:Tb; Y₂SiO₅:Ln (Ln=lanthanides); Y₂O₃:Ln (Ln=lanthanides); Y₂O₂S:Ln (Ln=lanthanides); YVO₄:A (A=lanthanides, In); Y(P,V)O₄:Eu; YTaO₄:Nb; YAlO₃:A (A=Pr, Tm, Er, Ce); YOCl:Yb,Er; LnPO₄:Ce,Tb (Ln=lanthanides or mixtures of lanthanides); LuVO₄:Eu; GdVO₄:Eu; Gd₂O₂S:Tb; GdMgB₅O₁₀:Ce,Tb; LaOBr:Tb; La₂O₂S:Tb; LaF₃:Nd,Ce; BaYb₂F₈:Eu; NaYF₄:Yb,Er; NaGdF₄:Yb,Er; NaLaF₄:Yb,Er; LaF₃:Yb,Er,Tm; BaYF₅:Yb,Er; Ga₂O₃:Dy; GaN:A (A=Pr, Eu, Er, Tm); Bi₄Ge₃O₁₂; LiNbO₃:Nd,Yb; LiNbO₃:Er; LiCaAlF₆:Ce; LiSrAlF₆:Ce; LiLuF₄:A (A=Pr, Tm, Er, Ce); Li₂B₄O₇:Mn, SiOₓ:Er,Al (0<x<2).

Particular preference is given to using the following materials as lid nanoparticles:

YVO₄:Eu, YVO₄:Sm, YVO₄:Dy, LaPO₄:Eu, LaPO₄:Ce, LaPO₄:Ce,Tb, LaPO₄:Ce,Dy, LaPO₄:Ce,Nd, ZnS:Tb, ZnS:TbF₃, ZnS:Eu, ZnS:EuF₃, Y₂O₃:Eu, Y₂O₂S:Eu, Y₂SiO₅:Eu, SiO₂:Dy, SiO₂:Al, Y₂O₃:Tb, CdS:Mn, ZnS:Tb, ZnS:Ag or ZnS:Cu. From the particularly preferred materials, in particular those having a cubic host lattice structure are selected, since the number of individual fluorescent bands reaches a minimum in these materials. Examples of these are: MgF₂:Mn; ZnS:Mn, ZnS:Ag, ZnS:Cu, CaSiO₃:Ln, CaS:Ln, CaO:Ln, ZnS:Ln, Y₂O₃:Ln, or MgF₂:Ln (Ln=lanthanides).

The simple detection probe contains luminescent inorganic doped nanoparticles (lid nanoparticles) which can be detected, after excitation using a radiation source, by absorption and/or scattering and/or diffraction of the exciting radiation or by emission of fluorescent light and whose surface is prepared in such a way that affinity molecules can couple to said prepared surface in order to detect a biological or other organic substance.

The surface preparation may be such that the surface of the lid nanoparticles is chemically modified and/or has reactive groups and/or covalently or noncovalently bound linker molecules.

An example of a chemical modification of the surface of the lid nanoparticle which may be mentioned is the coating of the lid nanoparticle with silica: silica enables a simple chemical conjugation of organic molecules, since silica reacts very readily with organic linkers such as, for example, triethoxysilanes or chlorosilanes.

Another possibility for preparing the surface of the lid nanoparticles is to convert the oxidic transition metal compounds of which the lid nanoparticles are composed into the corresponding oxychlorides using chlorine gas or organic chlorinating agents. These oxychlorides react in turn with nucleophiles such as, for example, amino groups, to give transition metal nitrogen compounds. In this way it is possible, for example, to achieve direct conjugation of proteins via the amino groups of lysine side chains. After surface modification with oxychlorides, proteins may also be conjugated by using a bifunctional linker such as maleimidopropionic acid hydrazide.

In this connection, particularly useful molecules for noncovalent linkages are chain-like molecules with a polarity or charge opposite to that of the lid nanoparticle surface. Examples of linker molecules noncovalently linked to the lid nanoparticles which may be mentioned are anionic, cationic or zwitterionic detergents, acidic or basic proteins, polyamines, polyamides and polysulfonic or polycarboxylic acids. Said molecules can be adsorbed to the surface of the lid nanoparticle by simple coincubation. Binding of an affinity molecule to these noncovalently bound linker molecules may then be carried out using standard methods of organic chemistry, such as oxidation, halogenation, alkylation, acylation, addition, substitution or amidation of the adsorbed or adsorbable material. These methods for binding an affinity molecule to the noncovalently bound linker molecule may be applied to the linker molecule either prior to adsorption to the lid nanoparticle or after said linker molecule has already been adsorbed to the lid nanoparticle.

Not only can the surface of the lid nanoparticles have reactive groups but the attached linker molecules may, for their part, also have reactive groups which may serve as points of attachment to the surface of the lid nanoparticle or to further linker molecules or affinity molecules. Such reactive groups which may be charged or uncharged or which may have partial charges may be both located on the surface of the lid nanoparticles and be part of the linker molecules. Possible reactive functional groups may be amino groups, carboxylic acid groups, thiols, thioethers, disulfides, imidazoles, guanidines, hydroxyl groups, indoles, vicinal diols, aldehydes, alphahaloacetyl groups, N-maleimides, mercury organyls, aryl halides, acid anhydrides, isocyanates, isothiocyanates, sulfonyl halides, imido esters, diazoacetates, diazonium salts, 1,2-diketones, alpha-beta-unsaturated carbonyl compounds, phosphonic acids, phosphoric esters, sulfonic acids, azolides or derivatives of said groups.

Nucleic acid molecules may also serve as linker molecules. They form the linkage to an affinity molecule which in turn contains nucleic acid molecules with sequences complementary to the linker molecules.

The present invention further relates to providing an extended detection probe which comprises a combination of the simple detection probe with one or more affinity molecules or with a plurality of affinity molecules coupled to one another. These affinity molecules or the combination of different affinity molecules are selected based on their specific affinity for the biological substance, in order to be able to detect the presence or absence thereof. In this connection, any molecule or any combination of molecules can be used as affinity molecules which, on the one hand, can be conjugated to the simple detection probes and, on the other hand, specifically attach to the biological or other organic substance to be detected. The individual components of a combination of molecules may be applied to the simple detection probes simultaneously or successively.

In general it is possible to use those affinity molecules which are also utilized in the fluorescent organic dye molecules described in the prior art, in order to bind the latter specifically to the biological or other organic substance to be detected. An affinity molecule may be a monoclonal or polyclonal antibody, another protein, a peptide, an oligonucleotide, a plasmid or another nucleic acid molecule, an oligo- or polysaccharide or a hapten such as biotin or digoxin or a low molecular weight synthetic or natural antigen. A list of such molecules have been published in the generally accessible literature, for example in “Handbook of Fluorescent Probes and Research Chemicals” (7^{th} edition, CD-ROM) by R. P. Hauglund, Molecular Probes, Inc.

The affinity of the extended detection probe for the biological agent to be detected generally results from the simple detection probe being coupled to a, usually organic, affinity molecule which has the desired affinity for the agent to be detected. In this connection, reactive groups on the surface of the affinity molecule and of the simple detection probe are utilized in order to bind these two molecules covalently or noncovalently. Reactive groups on the surface of the affinity molecule are amino groups, carboxylic acid groups, thiols, thioethers, disulfides, imidazoles, guanidines, hydroxyl groups, indoles, vicinal diols, aldehydes, alpha-haloacetyl groups, N-maleimides, mercury organyls, aryl halides, acid anhydrides, isocyanates, isothiocyanates, sulfonyl halides, imido esters, diazoacetates, diazonium salts, 1,2 -diketones, alpha-beta-unsaturated carbonyl compounds, or azolides. The groups for conjugating the affinity molecule, described further above, may be used on the surface of the simple detection probe.

One of the many possibilities of linking a simple detection probe to a protein as affinity molecule, which may be mentioned, is the following reaction. A silica-coated lid nanoparticle reacts with 3-aminopropyltriethoxysilane (Pierce, Rockford, Ill., USA), followed by SMCC activation (succinimidyl 4-[N-maleimidomethyl]cyclohexane 1-carboxylate (Pierce). The protein-bound thiol groups required for reaction to this activated lid nanoparticle can be generated by reacting a lysine-containing protein with 2-iminothiolane (Pierce). In this reaction, lysine side chains of the protein to be conjugated react with 2-iminothiolane with ring opening and thioamidine formation. The thiol groups formed, which are covalently linked to the protein, are then able to react in a hetero-Michael addition with the maleimide groups conjugated on the surface of the simple detection probe, in order to form a covalent bond between the protein as affinity molecule and the simple detection probe.

Besides the abovementioned possibility of forming extended detection probes from affinity molecule and simple detection probes by coupling, there are countless other methods which can be derived from the known reactivity of numerous commercially available linker molecules.

Apart from covalent linkages between simple detection probe and affinity molecule, noncovalent, self-organized linkages can be produced. One possibility which may be mentioned here is the linkage of simple detection probes with biotin as linker molecule to avidin- or streptavidin-coupled affinity molecules.

Another noncovalent, self-organized linkage between simple detection probe and affinity molecule is the interaction of simple detection probes, containing nucleic acid molecules, with complementary sequences conjugated on the surface of an affinity molecule.

An extended detection probe may also be formed by nucleic acid sequences being directly bound to the prepared surface of a simple detection probe or forming the reactive group of an affinity molecule. An extended detection probe of this kind is used for detecting nucleic acid molecules having complementary sequences.

The present invention further relates to a method for preparing a simple detection probe, to a method for preparing the extended detection probe and to a method for detecting a particular substance in a biological material.

The method of the invention for preparing the simple detection probe comprises the following steps:

a) preparation of lid nanoparticles

b) chemical modification of the surface of said lid nanoparticles and/or

c) preparation of reactive groups on the surface of the lid nanoparticles and/or

d) linking one or more linker molecules to the surface of the lid nanoparticles by covalent or noncovalent binding.

The distribution range of the expansions of the lid nanoparticles prepared in step a) is preferably limited to a range of +/−20% of an average expansion.

The method of the invention for preparing the extended detection probe comprises the following steps:

e) providing the simple detection probe

f) modifying the surface of an affinity molecule in order to introduce reactive groups which permit conjugation to the linker molecule

g) conjugating the activated affinity molecule and the simple detection probe.

The inventive method for detecting a particular substance in a biological material comprises the steps:

h) combining the extended detection probe and the biological and/or organic material

i) removing extended detection probes which have not bound,

j) exposing the material to electromagnetic radiation or to a particle beam

k) measuring the fluorescent light or measuring the absorption and/or scattering and/or diffraction of the radiation or the change therein.

An analyte is detected in a biological material to be studied by contacting the extended detection probe with a material to be studied. The biological material to be studied may be serum, cells, tissue sections, cerebral spinal fluid, sputum, plasma, urine or any other sample of human, animal or plant origin.

In this connection, the analyte to be studied should preferably already be immobilized or should be capable of being immobilized in a simultaneous or consecutive formation of supermolecular assemblages. An example of those immobilizations is an ELISA (enzyme linked immunosorbent assay) in which the antigens to be detected are specifically attached to a solid phase via adsorbed or primary antibodies bound in some other way. The antigen to be detected can also be readily immobilized if it is contained in an existing cell assemblage such as a tissue section or in individual cells fixed to a support.

If the analyte immobilized in this way is contacted with the extended detection probes, the latter will specifically attach to said analyte via the affinity molecule which they contain. An excess of extended detection probes can be readily washed off, and only specifically bound extended detection probes remain in the sample to be studied. When irradiating the sample prepared in this way using a suitable energy source, the presence of the extended detection probe containing the lid particle can be detected by detecting the emitted fluorescent light or by measuring changes in the absorbed, scattered or diffracted radiation. Thus the presence of those biological and/or organic substances which have a suitable affinity for the extended detection probe is detected. In this way it is possible to qualitatively and quantitatively detect substances in an assay independently of their chemical nature, as long as another molecule having a sufficiently high affinity for them exists. The extended detection probes are specific in that such an affinity molecule which has a high specific binding constant for the biological substance to be detected is attached on the surface of the simple detection probes contained in said extended detection probes. In this way it is also possible to detect particular cell types (for example cancer cells). In this connection, cell type-specific biomolecules may be labeled with the detection probes on the cell surface or else inside the cell and optically detected via a microscope or via a flow cytometer.

According to the above-described detection principle, it is also possible to detect a plurality of different analytes simultaneously in a biological and/or organic material (multiplexing). This is carried out by contacting the biological and/or organic material to be studied with different detection probes at the same time. The different detection probes differ from one another in that their affinity molecules attach to different analytes and the lid nanoparticles contained in said detection probes absorb, scatter or diffract or emit fluorescent light at different wavelengths.

The detection probe of the invention is stable to the irradiated energy and stable to oxygen or free radicals. The material of which the detection probes of the invention are composed is nontoxic or only slightly toxic. A very narrow size distribution width of the lid nanoparticles is not necessary, since the spectral position of the fluorescent bands and the bandwidths thereof depend on the doping and do not substantially depend on the size of the lid nanoparticles. Likewise, no inorganic shell around the particles is required in order to stabilize the fluorescence yield. However, it may be used in order to facilitate the conjugation chemistry. Another advantage is the fact that excitation can be carried out using a single broadband or narrowband radiation source, since the absorption wavelength of the exciting radiation or the excitation wavelength of the particles is not correlated with the emission wavelength. Moreover, time-resolved fluorescence measurement allows separation of the specific fluorescent light from unspecific background fluorescence, since the lifetime of the lid-particle state which is excited by the exterior radiation source and which then leads to the emission of light is usually substantially longer than that of the background fluorescence.

The detection probe of the invention and the method of the invention are preferably used in medical diagnostics and in screening techniques, in particular where the labeling of specific substances for the purposes of their detection, their localization and/or their quantification plays a particular part. This includes the detection of specific antibodies in diagnostic assays which are carried out for blood or other body materials. The detection probes of the invention may, however, also be used in cellular analysis, i.e. for detecting specific cells such as cancer cells. The detection probes of the invention provide particular advantages for the possible uses mentioned, since here the possibility of multiplexing, i.e. the simultaneous detection of different antigens in one assay or even in a single cell, can be utilized.

### EXAMPLES

### Example 1

### Preparation of lid Nanoparticles Consisting of YVO₄:Ln

The first step is to provide YVO₄:Ln. YVO₄:Ln can be prepared by the method described in K. Riwotzki, M. Haase; Journal of Physical Chemistry B; Vol. 102, 1998, page 10130, left-hand column. 3.413 g of Y(NO₃)₃.6H₂O (8.9 mmol) and 0.209 g of Eu(NO₃)₃.6H₂O (0.47 mmol) are dissolved in 30 ml of distilled water in a Teflon container. 2.73 g of Na₃(VO₄).10H₂O dissolved in 30 ml of distilled water are added with stirring. After stirring for another 20 min, the Teflon container is placed in an autoclave and heated to 200° C. with further stirring. After 1 h, the dispersion is removed from the autoclave and centrifuged at 3000 g for 10 min. The solids portion is extracted and taken up in 40 ml of distilled water. 3220 g of an aqueous 1-hydroxyethane-1,1-diphosphonic acid solution (60% by weight) are added to the dispersion (9.38 mmol). Y(OH)₃ which has formed from excess yttrium ions is removed by adjusting the pH to 0.3 with HNO₃ and stirring for 1 h. This leads to the formation of colloidal V₂O₅ which is noticeable by a reddish color of the solution. The pH is then adjusted to 12.5 with NaOH and the solution is stirred in a closed container overnight. The resulting white dispersion is then centrifuged at 3000 g for 10 min and the supernatant containing its byproducts is removed. The precipitate consists of YVO₄:Eu and can be taken up in 40 ml of distilled water.

The nanoparticles which are smaller than approx. 30 nm are isolated by centrifuging the dispersion at 3000 g for 10 min, decanting the supernatant and putting it aside. The precipitate was then again taken up in 40 ml of distilled water, centrifuged at 3000 g for 10 min and the supernatant was decanted. This supernatant and the supernatant set aside were then combined and centrifuged at 60 000 g for 10 min. The supernatant resulting herefrom contains the desired particles. After a further dialysis step (dialysis tube Serva, MWCO 12-14 kD), a colloidal solution is obtained, from which a redispersible powder can be obtained by drying using a rotary evaporator (50° C.).

### Example 2

### Preparation of lid Nanoparticles Consisting of LaPO₄:Eu

The first step is to provide LaPO₄:Eu. LaPO₄:Eu can be prepared according to the method described in H. Meyssamy, K. Riwotzki, A. Kornowski, S. Naused, M. Haase; Advanced Materials, Vol. 11, Issue 10, 1999, page 843, right-hand column bottom to page 844, left-hand column top. 12.34 g of La(NO₃)₃.6H₂O (28.5 mmol) and 0.642 g of Eu(NO₃)₃.5H₂O (1.5 mmol) are dissolved in 50 ml of distilled water in a Teflon pot and added to 100 ml of NaOH (1M). A solution of 3.56 g (NH₄)₂HPO₄ (₂₇ mmol) in 100 ml of distilled water is added with stirring. The solution is adjusted to a pH of 12.5 with NaOH (4M) and heated at 200° C. in an autoclave with vigorous stirring for 2 h. The dispersion is then centrifuged at 3150 g for 10 min and the supernatant is removed. In order to remove undesired La(OH)₃, the precipitate is dispersed in HNO₃ (1M) and stirred for ₃ days (pH 1). The dispersion is then centrifuged (3150 g, 5 min) and the supernatant is removed. 40 ml of distilled water are added with stirring to the centrifugate.

The milky dispersion still contains a broad size distribution. In order to isolate the nanoparticles which are smaller than approx. 30 nm, appropriate centrifugation and decanting steps are added to the procedure, in complete analogy to Example 1.

### Example 3

### Preparation of lid Nanoparticles Consisting of LaPO₄:Ce,Tb

The first step is to provide LaPO₄:Ce,Tb. 300 ml of tris(ethylhexyl) phosphate are flushed in a dry nitrogen gas stream. Subsequently, 7.43 g of LaCl₃.7H₂O (20 mmol), 8.38 g of CeCl₃.7H₂O (22.5 mmol) and 2.8 g of TbCl₃.6H₂O (7.5 mmol) are dissolved in 100 ml of methanol and added. Then water and methanol are distilled off under reduced pressure by heating the solution at 30° C. to 40° C. A freshly prepared solution consisting of 4.9 g of crystalline phosphoric acid (50 mmol) which have been dissolved in a mixture of 65.5 ml of trioctylamine (150 mmol) and 150 ml of tris(ethylhexyl) phosphate are then added. The clear solution must quickly be placed in a vessel to be evacuated and must be flushed with a nitrogen gas stream in order to minimize oxidation of Ce³⁺ when the temperature is raised. The solution is then heated to 200° C. During the heating phase, some of the phosphoric ester groups are cleaved, leading to a gradual decrease in the boiling point. The heating phase is ended when the temperature drops to 175° C. (approx. 30 to 40 h). After the solution has been cooled to room temperature, a four-fold excess of methanol is added causing the nanoparticles to precipitate. The precipitate is removed, washed with methanol and dried.

### Example 4

### Preparation of lid Nanoparticles Consisting of LaPO₄:Eu

490 mg (5.0 mmol) of crystalline phosphoric acid and 6.5 ml (15 mmol) of trioctylamine are dissolved in 30 ml of tris(ethylhexyl) phosphate. Subsequently, 1.76 g of La(NO₃)₃.7H₂O (4.75 mmol) and 92 mg of EuCl₃.6H₂O (0.25 mmol) are dissolved in 50 ml of tris(ethylhexyl) phosphate and combined with the first solution. The resulting solution is degassed under reduced pressure and subsequently heated at 200° C. under nitrogen for 16 h. During the heating phase, some of the phosphoric ester groups are cleaved, leading to a gradual decrease in the boiling point. The heating phase is ended when the temperature drops to 180° C. After the solution has been cooled to room temperature, methanol is added causing the nanoparticles to precipitate. The precipitate is removed with the aid of a centrifuge, washed twice with methanol and dried.

### Example 5

### Coating of the Nanoparticles Prepared in Example 2 with an SiO₂ Coat

1 g of the LaPO₄:Eu nanoparticles prepared in example 2 is introduced into 100 ml of water with vigorous stirring using a magnetic stirrer at 900 rpm, and the mixture is adjusted to a pH of 12 with tetrabutylammonium hydroxide. 500 mg of sodium water glass (26.9% SiO₂:8.1% Na₂O) are then added to the dispersion with vigorous stirring. This is followed by adding dropwise, also with vigorous stirring, 50 ml of ethanol. The resultant precipitate is removed by centrifugation and the residue is redispersed in 50 ml of deionized water in an ultrasound bath. The dispersion is then separated from the undispersed portion by decanting and dried with the aid of a rotary evaporator at a pressure of 10 mbar and a temperature of 80° C.

### Example 6

### Conjugation of the Nanoparticles Prepared in Example 5 with Anti-α-actin Antibodies

100 mg of the silica-coated nanoparticles synthesized in Example 5 are dispersed in 10 ml of dry tetrahydrofuran (THF) and mixed with 100 μl of N-methylmorpholine. 0.5 ml of a 10% strength solution of 3-aminopropyltriethoxysilane in THF is added to the solution. The solution is stirred at 40° C. in a tightly sealed vessel overnight. The solution is mixed with 5 ml of water, stirred at room temperature for a further 1 h and material that may have precipitated is filtered off using a glass fritt (4G, Schott). The filtrate is buffered in TSE7 buffer (TSE7: 100 mmol of triethanolamine, 50 mmol of sodium chloride, 1 mmol of EDTA in water, pH 7.3) in ultrafiltration tubes (Centricon, Amicon, cut-off 10, kD). The target volume is 2 ml, the exchange factor is 1000. The amino-activated nanoparticles retained by the ultrafiltration membrane in 2 ml of TSE7 buffer are admixed with 500 μl of a solution of 20 mmol of sSMCC (sulfosuccimidyl 4-[N-maleimidomethyl]cyclohexane carboxylate (Pierce, Rockwell, Ill., USA) and stirred at 25° C. for 60 minutes. The mixture obtained is buffered in TSE7 buffer in 10 kD Centricon tubes, as described above, and the volume is reduced to 2 ml. This step is carried out at 5° C. The solution obtained is stable in a refrigerator for 12 h. 10 mg of monoclonal anti-actin antibody (Sigma) are transferred into a TSE8 buffer (exchange factor 1000, TSE8: (100 mmol of triethanolamine, 50 mmol of sodium chloride, 1 mmol of EDTA in water, pH 8.5) by means of Centricon ultrafiltration tubes (cut-off 50 kD). The protein concentration is adjusted to 7-8 mg/ml. 150 μl of a 10 mM solution of 2-iminothiolane in TSE8 buffer are added to the antibody solution and the mixture is left reacting for 15 minutes. The thus thiol-activated antibody is buffered with TSE8 buffer at 4° C., as described above, in order to remove unreacted activator molecules and the volume is reduced to 2 ml. The activated nanoparticles and the solutions containing the activated antibody are combined and stirred at room temperature overnight. The thus obtained dispersion of the extended detection particle is relieved of unreacted antibody by gel permeation chromatography on Superdex 200 (Pharmacia). The running buffer used is TSE7. The retention time of the unconjugated extended lid nanoparticle is approximately 2 hours.

### Example 7

### Conjugation of lid Nanoparticles Prepared in Example 1 with Antimyoglobin Antibodies

100 mg of the vanadate nanoparticles prepared in Example 1 are heated in a glass tube in an argon stream using a heating tape. After baking out the particles for approximately one hour, 3-5% by volume of chlorine gas are metered into the gas stream for approximately 3 min. The reaction time required depends substantially on the particle size and should therefore be determined by titration possibly using the same batch of nanoparticles. The particles are left cooling in the argon stream and the partially chlorinated nanoparticles are added to 5 ml of a 50 mM solution of N-maleimidopropionic acid hydrazide (Pierce), and the solution is stirred at 20° C. overnight. The solution obtained in this way is concentrated at room temperature in a rotary evaporator and, as described in Example 6, buffered in TSE7 using ultrafiltration tubes. The dispersion obtained is stable at 5° C. for 12 h. 10 mg of polyclonal rabbit anti-myoglobin antibody (Dako) are transferred into TSE8 buffer (exchange factor 100, TSE8: (100 mmol of triethanolamine, 50 mmol of sodium chloride, 1 mmol of EDTA in water, pH 8.5) using Centricon ultrafiltration tubes (cut-off 50 kD). The protein concentration is adjusted to 7-8 mg/ml. 150 μl of a 10 mM solution of 2-iminothiolane in TSE8 buffer are added to the antibody solution and the mixture is left reacting for 15 minutes. The thus thiol-activated antibody is buffered with TSE8 buffer at 4° C., as described above, in order to remove unreacted activator molecules and the volume is reduced to 2 ml. The activated nanoparticles and the solutions containing the activated antibody are combined and stirred at room temperature overnight. The thus obtained dispersion of the extended detection particle is relieved of unreacted antibody by gel permeation chromatography on Superdex 200 (Pharmacia). The running buffer used is TSE7. The retention time of the extended lid nanoparticle is approximately 2 hours.

### Example 8

### Visualization of Actin Filaments in Rabbit Muscle Cells via Nanoparticle Fluorescence

A thin section of a rabbit muscle, cut using a freezing microtome, is applied to a slide and fixed in ice-cold ethanol for 3 minutes. The thin section applied to the slide is then washed twice with PBS-Tween buffer (137 mM NaCl, 2.7 mM KCl, 10.1 mM Na2HPO4, 1.8 mM, KH2PO4, 0.1% Tween 20), and in each case left in the washing buffer for 5 min. Nonspecific binding is reduced by incubating the thin section in a solution of 1.5% sheep serum in PBS-Tween buffer at 20° C. for 30 min, and the thin section is washed twice as described above. The solution of the extended detection particle, described in Example 6, is diluted 1:100 with PBS-S (137 mM NaCl, 2.7 mM KCl, 10.1 mM Na2HPO4, 1.8 mM, KH2PO4, 0.1% Tween 20, 1.5% sheep serum), and the thin section is incubated in this solution at 20° C. for one hour. The incubation is followed by washing the thin section as described above. Detection is carried out, after excitation at wavelengths between 333 nm and 364 nm using an argon ion laser, by measuring the fluorescent light at a wavelength of 591 nm. For this, a confocal laser scanning microscope, type TCS NT from Leica, was used.

### Example 9

### Detection of Myoglobin in Human Serum via Nanoparticle Fluorescence

Monoclonal anti-myoglobin antibodies (BiosPacific) are dissolved at a concentration of 5 mg/L in C buffer (100 mmol of sodium carbonate in water, pH 9.0). 200 μl of this solution are pipetted into each of 96 wells of a standard polystyrene ELISA plate (Greiner), and the plate is sealed and incubated at 37° C. for 2 h. The plate is tapped out and blocked with 200 μl of a 1% BSA (bovine serum albumin) solution in TSE7 buffer (see Example 6). The plate is washed three times with in each case 250 μl of TSET7 buffer (100 mmol of triethanolamine, 50 mmol of sodium chloride, 1 mmol of EDTA, 0.1% Tween 20 in water, pH 7.3). In each case 100 μl of a 6-level myoglobin calibrator (Bayer Immuno 1) and human sera are pipetted into the different wells of the microtiter plate and incubated at room temperature for 2 h. The analyte solutions are removed from the plate by pipetting and the plate is washed three times as described above. Approximately 1 μg of the extended anti-myoglobin detection probe prepared in Example 7 and dispersed in 100 μl of TSE7 is added to each well. This is followed by incubating at room temperature for 1 h and washing three times with TSET7. The ELISA is read out by measuring the lid nanoparticle fluorescence in a microtiter plate reader (Tecan).

### Example 10

### Dissolving the Nanoparticles Prepared in Example 3 in Water by Reacting Ethylene Glycol or Polyethylene Glycol

1 g of the LaPO₄:Ce,Tb (˜5 mmol) prepared in Example 3 is heated together with 100 ml of ethylene glycol (˜2 mol) (alternatively, polyethylene glycols of varying chain length, HO—(CH₂—CH₂—O)ₙ—OH, where n=2-9, may also be used) and 100 mg of paratoluene sulfonic acid to 200° C. with stirring and nitrogen. In the process, the particles dissolve and remain in solution even after cooling to room temperature. This is followed by dialysis against water overnight (cut-off MW 10-20.000).

### Example 11

### Functionalization of Nanoparticles Prepared in Example 10 by Oxidation

Firstly, 0.5 ml of 96-98% strength sulfuric acid is added with stirring to 100 mg (0.5 mmol in 20 ml of water) of the nanoparticles prepared in Example 10.1 mM KmnO₄ solution is added dropwise until the purple color no longer disappears. Subsequently, the same amount of KmnO₄ solution is added again and the solution is left stirring at room temperature overnight (>12 h). Excess permanganate is reduced by adding freshly prepared 1 mM sodium sulfite solution dropwise. This is followed by dialysis against 0.1M MES, 0.5M NaCl, pH 6.0 overnight.

### Example 12

### Conjugation of Nanoparticles Prepared in Example 11 to Anti-Biotin Antibodies

0.4 mg of EDC (˜2 mM) and 1.1 mg (˜5 mM) of sulfo-NHS (both from Pierce; Rockford, Ill.) are added 1 mg (5 nmol) of the carboxy-functionalized nanoparticles prepared in Example 11 in 1 ml of buffer (0.1 M MES, 0.5 M NaCl, pH 6) and the solution is stirred at room temperature for 15 min. The unreacted EDC is inactivated by adding 1.4 μl of 2-mercaptoethanol (final concentration 20 mM). The same molar amount (5 nmol) of polyclonal goat antibiotin antibody (Sigma) in activation buffer (0.1M MES, 0.5M NaCl, pH 6.0) is added and the mixture is stirred at room temperature for 2 h. The reaction is stopped by adding hydroxylamine (final concentration 10 mM). The thus obtained solution of the extended detection particles is relieved of unreacted antibody by gel permeation chromatography on Superdex 200 (Pharmacia). The running buffer used is activation buffer. The retention time of the extended lid nanoparticle is approximately 2 hours.

1. A simple detection probe containing luminescent inorganic doped nanoparticles (lid nanoparticles) which can be detected, after excitation using a radiation source, by absorption and/or scattering and/or diffraction of the exciting radiation or by emission of fluorescent light and whose surface is prepared in such a way that affinity molecules can couple to said prepared surface in order to detect a biological or other organic substance.

2. The simple detection probe as claimed in claim 1, characterized in that the surface of the lid nanoparticles is chemically modified and/or has covalently or noncovalently bound linker molecules and/or reactive groups.

3. The simple detection probe as claimed in claim 2, characterized in that the chemical modification of the surface involves a coat of silica around the surface of the lid nanoparticle.

4. The simple detection probe as claimed in claim 2, characterized in that the chemical modification involves oxychlorides which is generated by treating lid nanoparticles composed of oxidic transition metal compounds with chlorine gas or organic chlorinating agents.

5. The simple detection probe as claimed in any of claims 2 to 4, characterized in that one or more chain-like molecules with a polarity or charge opposite to that of the lid nanoparticle surface are noncovalently linked as linker molecule to the surface of the lid nanoparticles.

6. The simple detection probe as claimed in claim 5, characterized in that the chain-like molecules are anionic, cationic or zwitterionic detergents, acidic or basic proteins, polyamines, polyamides or polysulfonic or polycarboxylic acids.

7. The simple detection probe as claimed in any of claims 2, 5 or 6, characterized in that the surface and/or the linker molecules linked to the lid nanoparticle surface carry reactive neutral, charged or partially charged groups such as amino groups, carboxylic acid groups, thiols, thioethers, disulfides, imidazoles, guanidines, hydroxyl groups, indoles, vicinal diols, aldehydes, alpha-haloacetyl groups, N-maleimides, mercury organyls, aryl halides, acid anhydrides, isocyanates, isothiocyanates, sulfonyl halides, imido esters, diazoacetates, diazonium salts, 1,2-diketones, alpha-beta-unsaturated carbonyl compounds, azolides, phosphonic acids, phosphoric esters, or derivatives of said groups, said reactive groups allowing chemical binding to further linker molecules or affinity molecules.

8. The simple detection probe as claimed in any of claims 2 to 4, characterized in that nucleic acid molecules serves as linker molecules for an affinity molecule containing nucleic acid molecules with sequences complementary to said linker molecules.

9. The simple detection probe as claimed in any of claims 1 to 8, characterized in that the radiation source is a source of electromagnetic radiation with wavelengths in the range of infrared light, of visible light, of UV, of X-ray light or of γ radiation or is a source of particle radiation such as electron radiation.

10. The simple detection probe as claimed in any of claims 1 to 9, characterized in that the lid nanoparticles have diameters in the range from 1 nm to 1 μm, preferably in the range from 2 nm to 100 nm, particularly preferably in the range from 2 nm to below 20 nm and very particularly preferably between 2 nm and 10 nm.

11. The simple detection probe as claimed in any of claims 1 to 9, characterized in that the lid nanoparticles have a needle-like morphology with a width of from 3 nm to 50 nm, preferably from 3 nm to below 20 nm, and a length of from 20 nm to 5 μm, preferably from 20 nm to 500 nm.

12. The simple detection probe as claimed in any of claims 1 to 11, characterized in that the host material of the lid nanoparticles comprises compounds of the XY type, X being a cation of one or more elements of the main groups 1a, 2a, 3a, 4a, of the transition groups 2b, 3b, 4b, 5b, 6b, 7b or of the lanthanides of the Periodic Table and Y being either a polyatomic anion of one or more element(s) of the main groups 3a, 4a, 5a, of the transition groups 3b, 4b, 5b, 6b, 7b and/or 8b and element(s) of the main groups 6a and/or 7 or a monoatomic anion of the main groups 5a, 6a or 7a of the Periodic Table.

13. The simple detection probe as claimed in claim 12, characterized in that the host material of the lid nanoparticles comprises compounds of the group consisting of sulfides, selenides, sulfoselenides, oxysulfides, borates, aluminates, gallates, silicates, germanates, phosphates, halophosphates, oxides, arsenates, vanadates, niobates, tantalates, sulfates, tungstates, molybdates, alkali halides and other halides or nitrides.

14. The. simple detection probe as claimed in any of claims 1 to 13, characterized in that one or more elements of the group comprising elements of the main groups 1a, 2a or Al, Cr, Tl, Mn, Ag, Cu, As, Nb, Nd, Ni, Ti, In, Sb, Ga, Si, Pb, Bi, Zn, Co and/or elements of the lanthanides are used as doping agent.

15. The simple detection probe as claimed in claim 14, characterized in that combinations of two or more of said elements at different concentrations relative to one another also serve as doping material.

16. The simple detection probe as claimed in any of claims 1 to 15, characterized in that the concentration of the doping material in the host lattice is between 10⁻⁵ mol % and 50 mol %, preferably between 0.01 mol % and 30 mol %, particularly preferably between 0.1 mol % and 20 mol %.

17. The simple detection probe as claimed in any of claims 1 to 16, characterized in that LiI:Eu; NaI:Tl; CsI:Tl; CsI:Na; LiF:Mg; LiF:Mg,Ti; LiF:Mg,Na; KMgF₃:Mn; Al₂O₃:Eu; BaFCl:Eu; BaFCl:Sm; BaFBr:Eu; BaFCl_{0.5}Br_{0.5}:Sm; BaY₂F₈:A (A=Pr, Tm, Er, Ce); BaSi₂O₅:Pb; BaMg₂Al₁₆O₂₇:Eu; BaMgAl₁₄O₂₃:Eu; BaMgAl₁₀O₁₇:Eu; BaMgAl₂O₃:Eu; Ba₂P₂O₇:Ti; (Ba,Zn,Mg)₃Si₂O₇:Pb; Ce(Mg,Ba)Al₁₁O₁₉; Ce_{0.65}Tb_{0.35}MgAl₁₁O₁₉:Ce,Tb; MgAl₁₁O₁₉:Ce,Tb; MgF₂:Mn; MgS:Eu; MgS:Ce; MgS:Sm; MgS:(Sm,Ce); (Mg,Ca)S:Eu; MgSiO₃:Mn; 3.5MgO.0.5MgF₂.GeO₂:Mn; MgWO₄:Sm; MgWO₄:Pb; 6MgO.As₂O₅:Mn; (Zn,Mg)F₂:Mn; (Zn₄Be)SO₄:Mn; Zn₂SiO₄:Mn; Zn₂SiO₄:Mn,As; ZnO:Zn; ZnO:Zn,Si,Ga; Zn₃(PO₄)₂:Mn; ZnS:A (A=Ag, Al, Cu); (Zn,Cd)S:A (A=Cu, Al, Ag, Ni); CdBO₄:Mn; CaF₂:Mn; CaF₂:Dy; CaS:A A=lanthanides, Bi); (Ca,Sr)S:Bi; CaWO₄:Pb; CaWO₄:Sm; CaSO₄:A (A=Mn, lanthanides); 3Ca₃(PO₄)₂.Ca(F,Cl)₂:Sb,Mₙ; CaSiO₃:Mn,Pb; Ca₂Al₂Si₂O₇:Ce; (Ca,Mg)SiO₃:Ce; (Ca,Mg)SiO₃:Ti; 2SrO.6(B₂O₃).SrF₂:Eu; 3Sr₃(PO₄)₂.CaCl₂:Eu; A₃(PO₄)₂.ACl₂:Eu (A=Sr, Ca, Ba); (Sr,Mg)₂P₂O₇:Eu; (Sr,Mg)₃(PO₄)₂:Sn; SrS:Ce; SrS:Sm,Ce; SrS:Sm; SrS:Eu; SrS:Eu,Sm; SrS:Cu,Ag; Sr₂P₂O₇:Sn; Sr₂P₂O₇:Eu; Sr₄Al₁₄O₂₅:Eu; SrGa₂S₄:A (A=lanthanides, Pb); SrGa₂S₄:Pb; Sr₃Gd₂Si₆O₁₈:Pb,Mn; YF₃:Yb,Er; YF₃:Ln (Ln=lanthanides); YLiF₄:Ln (Ln=lanthanides); Y₃Al₅O₁₂:Ln (Ln=lanthanides); YAl₃(BO₄)₃:Nd,Yb; (Y,Ga)BO₃:Eu; (Y,Gd)BO₃:Eu; Y₂Al₃Ga₂O₁₂:Tb; Y₂SiO₅:Ln (Ln=lanthanides); Y₂O₃:Ln (Ln=lanthanides); Y₂O₂S:Ln (Ln=lanthanides); YVO₄:A (A=lanthanides, In); Y(P,V)O₄:Eu; YTaO₄:Nb; YAlO₃:A (A=Pr, Tm, Er, Ce); YOCl:Yb,Er; LnPO₄:Ce,Tb (Ln=lanthanides or mixtures of lanthanides); LuVO₄:Eu; GdVO₄:Eu; Gd₂O₂S:Tb; GdMgB₅O₁₀:Ce,Tb; LaOBr:Tb; La₂O₂S:Tb; LaF₃:Nd,Ce; BaYb₂F₈:Eu; NaYF₄:Yb,Er; NaGdF₄:Yb,Er; NaLaF₄:Yb,Er; LaF₃:Yb,Er,Tm; BaYF₅:Yb,Er; Ga₂O₃:Dy; GaN:A (A=Pr, Eu, Er, Tm); Bi₄Ge₃O₁₂; LiNbO₃:Nd,Yb; LiNbO₃:Er; LiCaAlF₆:Ce; LiSrAlF₆:Ce; LiLuF₄:A (A=Pr, Tm, Er, Ce); Li₂B₄O₇:Mn, SiOₓ:Er,Al (0<x<2) is used as material for the lid nanoparticles.

18. The simple detection probe as claimed in any of claims 1 to 16, characterized in that YVO₄:Eu, YVO₄:Sm, YVO₄:Dy, LaPO₄:Eu, LaPO₄:Ce, LaPO₄:Ce,Tb, LaPO₄:Ce,Dy, LaPO₄:Ce,Nd, ZnS:Tb, ZnS:TbF₃, ZnS:Eu, ZnS:EuF₃, Y₂O₃:Eu, Y₂O₂S:Eu, Y₂SiO₅:Eu, SiO₂:Dy, SiO₂:Al, Y₂O₃:Tb, CdS:Mn, ZnS:Tb, ZnS:Ag or ZnS:Cu is used as material for the lid nanoparticles.

19. The simple detection probe as claimed in any of claims 1 to 18, characterized in that material having a cubic host lattice structure is used for the lid nanoparticles.

20. The simple detection probe as claimed in any of claims 1 to 16, characterized in that MgF₂:Mn; ZnS:Mn, ZnS:Ag, ZnS:Cu, CaSiO₃:Ln, CaS:Ln, CaO:Ln, ZnS:Ln, Y₂O₃:Ln, or MgF₂:Ln (Ln=lanthanides) is used as material for the lid nanoparticles.

21. An extended detection probe for biological applications comprising a combination of the simple detection probe as claimed in any of claims 1 to 20 with one or more affinity molecules or a plurality of affinity molecules coupled to one another, it being possible for said affinity molecules on the one hand to attach to the prepared surface of the simple detection probe and on the other hand to bind to a biological or other organic substance.

22. The extended detection probe as claimed in claim 21, characterized in that the affinity molecules are monoclonal or polyclonal antibodies, proteins, peptides, oligonucleotides, plasmids, nucleic acid molecules, oligo- or polysaccharides, haptens such as biotin or digoxin or a low molecular weight synthetic or natural antigen.

23. The extended detection probe as claimed in claim 21 or 22, characterized in that the affinity molecule is coupled covalently or noncovalently to the simple detection probe via reactive groups on the affinity molecule and on the simple detection probe.

24. The extended detection probe as claimed in claim 23, characterized in that the reactive groups on the affinity molecule surface are amino groups, carboxylic acid groups, thiols, thioethers, disulfides, imidazoles, guanidines, hydroxyl groups, indoles, vicinal diols, aldehydes, alpha-haloacetyl groups, N-maleimides, mercury organyls, aryl halides, acid anhydrides, isocyanates, isothiocyanates, sulfonyl halides, imido esters, diazoacetates, diazonium salts, 1,2-diketones, alpha-beta-unsaturated carbonyl compounds, or azolides.

25. The extended detection probe as claimed in any of claims 21 to 23, characterized in that there is a noncovalent self-organized linkage between the simple detection probe and the affinity molecule.

26. The extended detection probe as claimed in claim 25, characterized in that there is a linkage between biotin as linker molecule of the simple detection probe and avidin or streptavidin as reactive group of the affinity molecule.

27. The extended detection probe as claimed in claim 26, characterized in that there is a linkage between nucleic acid molecules as linker molecules of the simple detection probe and nucleic acid molecules, having sequences complementary thereto, as reactive group of the affinity molecule.

28. The extended detection probe as claimed in claim 22, characterized in that nucleic acid sequences serve as affinity molecule and the biological substance to be detected comprises nucleic acid molecules with complementary sequences.

29. A method for preparing a simple detection probe as claimed in any of claims 1 to 20, comprising the steps
a) preparation of lid nanoparticles
b) chemical modification of the surface of said lid nanoparticles and/or
c) preparation of reactive groups on the surface of said lid nanoparticles and/or
d) linking one or more linker molecules with the surface of said lid nanoparticles by covalent or noncovalent binding.

30. The method for preparing the simple detection probe as claimed in claim 29, characterized in that the distribution range of the expansions of the lid nanoparticles prepared in step a) is limited to a range of +/−20% of an average expansion.

31. A method for preparing the extended detection probe as claimed in claims 21 to 28, comprising the steps
e) providing the simple detection probe
f) modifying the surface of an affinity molecule in order to introduce reactive groups which allow conjugation to the simple detection probe
g) conjugating the activated affinity molecule and the simple detection probe.

32. A method for detecting a biological or other organic substance, comprising the steps
h) combining the extended detection probe as claimed in any of claims 21 to 28 and the biological and/or organic material,
i) removing extended detection probes which have not bound,
j) exposing the sample to electromagnetic radiation or to a particle beam
k) measuring the fluorescent light or measuring the absorption and/or scattering and/or diffraction of the radiation or the change therein.

33. The method as claimed in claim 32, characterized in that the biological material to be studied is serum, cells, tissue sections, cerebral spinal fluid, sputum, plasma, urine or another sample of human, animal or plant origin.

34. The method as claimed in claim 32 or 33, characterized in that the analyte to be studied is immobilized in the biological or other material to be studied.

35. The method as claimed in any of claims 32 to 34, characterized in that the biological and/or organic material to be studied is combined with different extended detection probes at the same time, and said different extended detection probes differ from one another in that their affinity molecules attach to different analytes and the lid nanoparticles contained in said extended detection probes absorb, scatter or diffract or emit fluorescent light at different wavelengths.
